(19) United States
(12) Patent Application Publication
(10) Pub. No.: US 2025/0331907 A1
(43) Pub. Date: Oct. 30, 2025

(54) ACTIVELY STEERABLE SPHINCTEROTOME

(71) Applicant: Boston Scientific Medical Device Limited, Galway (IE)

(72) Inventors: Deepak Kumar Sharma, Muzaffarnagar (IN); Sharath Kumar G, Kanakapura (IN)

(73) Assignee: Boston Scientific Medical Device Limited, Galway (IE)

(21) Appl. No.: 19/188,872

(22) Filed: Apr. 24, 2025

Related U.S. Application Data
(60) Provisional application No. 63/638,816, filed on Apr. 25, 2024.

(51) Int. Cl. A61B 18/00; A61B 1/005; A61B 1/018

(52) U.S. Cl. CPC A61B 18/00; A61B 1/005; A61B 1/018; A61B 2018/00184; A61B 2018/00482; A61B 2018/00601; A61B 2018/0091; A61B 2018/00982

(57) ABSTRACT

Devices, systems, and methods for a steerable sphincterotome. A handle includes first and second portions connected respectively to a steering wire and wire filament. A distal tip includes a base component and a mobile component selectively articulated by the steering wire. The wire filament can be selectively articulated and deployed for cutting.

### CROSS-REFERENCE TO RELATED APPLICATIONS

This application claims the benefit of U.S. Provisional Patent Application Ser. No. 63/638,816 filed on Apr. 25, 2024, the disclosure of which is incorporated herein by reference.

### FIELD

The disclosure relates generally to devices, systems, and methods for steerable medical devices. More specifically, aspects of the disclosure pertain to devices, systems, and/or methods for surgical tomes.

### BACKGROUND

Endoscopic retrograde cholangiopancreatography (ERCP) may be used to diagnose and treat conditions of the common bile duct, including, for example, gallstones, inflammatory strictures, leaks (e.g., from trauma, surgery, etc.), and cancer. In an ERCP procedures, a physician may view, through an endoscope, the inside of the stomach and/or the duodenum. Often, dyes may be injected (e.g., via a lumen of a sphincterotome or other device) into the ducts in the biliary tree and pancreas so that the area can be seen using X-rays. These procedures may necessitate gaining and keeping access to the papilla of Vater, the common bile duct, and/or the pancreatic duct, which may be technically challenging, may require extensive training and practice to gain proficiency, and may require one or more expensive tools in order to perform.

During an ERCP procedure, a number of steps are typically performed while the patient is often sedated and/or anaesthetized. For example, an endoscope or duodenoscope may be inserted through the mouth, down the esophagus, into the stomach, through the pylorus into the duodenum, to a position at or near the papilla of Vater (also referred to as the ampulla of Vater), which is the opening of the common bile duct and the pancreatic duct. Due to the shape of the papilla, and the angle at which the common bile and pancreatic ducts meet the wall of the duodenum, the distal end of the endoscope or duodenoscope is generally placed just past the papilla. Due to this positioning beyond the papilla, the endoscopes or duodenoscopes typically used in these procedures are usually side-viewing devices. The side-viewing feature provides imaging along the lateral aspect of the tip rather than from the end of the endoscope. Such orientation may allow a clinician to obtain an image of the medial wall of the duodenum, where the papilla of Vater is located, even though the distal tip of the viewing device is beyond the opening. Once the papilla or a target area in the duodenum is visually located, an accessory medical device, such as sphincterotome or other accessory medical device, may be extended out a side opening or window of the endoscope or duodenoscope for facilitating access through the papilla and into a desired one of the common bile duct and the pancreatic duct.

Although not required, a fluid (e.g., a compressible fluid, such as compressible gas, air, nitrogen, carbon dioxide, etc., and/or other suitable fluid) may be delivered via catheter (e.g., as inserted through a subject as discussed above or otherwise inserted), a sphincterotome, and/or other elongated tubular device extending through an inserted endoscope, and/or other suitable elongate medical device to an opening (e.g., the papilla of Vater) of a body lumen of a subject. Applying the fluid to the opening may facilitate identifying and/or accessing the body lumen by at least partially dilating the opening with the fluid prior to and/or during delivery of a catheter, sphincterotome, guidewire, etc. to a target anatomical structure or tissue (e.g., a duct or other suitable structure or tissue) at or distal of the opening of the body lumen. Some example techniques and systems for cannulating an opening of a body lumen using fluid are described in U.S. Pat. No. 11,647,899, issued on May 16, 2023, and titled DEVICES, SYSTEMS AND METHOD FOR ACCESSING A BODY LUMEN, which is hereby incorporated by reference.

Alternatively or in addition to using fluid to open the opening of the body lumen, the opening may be manually opened by engaging a distal end or tip of an accessory medical device with the papilla. Further, the accessory medical device may be manipulated and/or the endoscope through which the accessory medical device extends may be manipulated to direct the accessory medical device through the papilla and into a desired one of the common bile duct and the pancreatic duct. Some example techniques and systems for manipulating a distal tip of a medical device, such as a guidewire, to access a desired body lumen are described in U.S. Patent Application Publication No. 2023/0372007, which was filed on May 18, 2023, and titled MEDICAL DEVICE WITH A STEERABLE TIP, which is hereby incorporated by reference.

Turning to the Figures, FIGS. 1 and 2 depict a selective cannulation during an ERCP procedure, which includes a guidewire 12 and/or an accessory medical device 14 (e.g., an endoscopic accessory device, such as a sphincterotome, and/or other suitable accessory medical device) being passed towards, against, and/or through a body lumen such as the major papilla 16 (e.g., ampullary entry) near the descending duodenum 18 to access the Sphincter of Oddi Complex 20. During the cannulation procedure, a distal portion of a medical device 22 (e.g., an endoscope, duodenoscope, guide catheter, etc.) may be positioned within the descending duodenum 18. The guidewire 12 and the accessory medical device 14 may be advanced through a working channel (e.g., a lumen) of the medical device 22 towards the major papilla 16. Additionally, the guidewire 12 and/or the accessory medical device 14 may be advanced against and/or through the major papilla 16 to one of the common bile duct 17 and the pancreatic duct 19.

Accessing the papilla 16, the common bile duct 17, and/or the pancreatic duct 19 may be difficult because the openings are small compared to a size (e.g., a diameter or other size measurement) of many accessory medical devices, the openings may be closely located, the openings may be completely collapsed/closed, the openings may extend into the descending duodenum 18 at an angle that may be difficult to visualize and/or access, and/or it may be difficult to control a position of a distal tip of the accessory medical device 14. Thus, a medical professional may be required to manipulate the accessory medical device 14 and guidewire 12 by manually rotating the medical device 22, pulling on a wire filament 30 to adjust the accessory medical device 14 in a single direction, and/or use an elevator and/or other suitable ramped surface within the distal end of the medical device 22 in an attempt to better align or orient the accessory medical device 14 and/or the guidewire 12 with respect to the medical device 22 and the openings of the papilla 16, the common bile duct 17, and/or the pancreatic duct 19. Difficult cannulation procedures in which the medical professional fails to access the body lumen within a certain time limit or after a certain number of unsuccessful attempts may lead to significant post-procedure complications, such as post-ERCP pancreatitis (PEP).

A medical professional accessing a body lumen (e.g., a duct, a papilla, a common bile duct, a pancreatic duct, or the like) by manipulating an accessory medical device against or into the opening of the body lumen, as discussed above, may subject the walls of the opening of the body lumen to compressive forces. Compression of a body lumen opening or the body lumen itself can cause buckling in what may be described as the “accordion effect”. As such, it is desirable to have control over a distal tip of the accessory medical device 14 such that a user may precisely access an opening of the papilla 16 and once through the papilla 16, direct the accessory medical device 14 and/or guidewire 12 to a desired one of the common bile duct 17 and the pancreatic duct 19.

### SUMMARY

This disclosure provides design, material, manufacturing method, and use alternatives for medical devices and medical systems. In a first example, a medical device includes an elongate tube having a proximal end and a distal end configured to be directed toward an opening of a body lumen; a wire filament extending along the elongate tube, a distal end of the wire filament connected to the distal end of the elongate tube, a proximal portion of the wire filament extending at least partially along the elongate tube, and a distal portion of the wire filament extending external to the elongate tube; a steerable distal tip extending distal of a distal end of the wire filament, the distal tip comprising a base component and a mobile component distal from the base component; a handle in mechanical communication with the steerable distal tip; and a steering wire extending from the handle to the mobile component of the steerable distal tip, such that adjustment of the handle causes tension in the steering wire to adjust the mobile portion of the distal tip relative to the base component of the distal tip.

Alternatively or additionally to any of the examples above, the handle can include a first actuating portion connected to the wire filament.

Alternatively or additionally to any of the examples above, actuation of the first actuating portion in an axial direction can be configured to cause the distal end of the elongate tube to flex and expose a cutting portion of the wire filament.

Alternatively or additionally to any of the examples above, the handle can include a second actuating portion connected to the steering wire.

Alternatively or additionally to any of the examples above, actuation of the actuating portion in an axial direction can adjust the position of the mobile portion of the distal tip relative to the base component of the distal tip.

Alternatively or additionally to any of the examples above, the steering wire can extend through at least a portion of the elongate tube. The elongate tube can include a first lumen for receiving the wire filament and a second lumen configured to receive the steering wire.

Alternatively or additionally to any of the examples above, adjustment of the handle in a radial direction relative to the elongate tube can be configured to adjust the position of the steerable distal tip.

Alternatively or additionally to any of the examples above, the steerable distal tip can further include a flexible tube connecting a lumen within the base component to a lumen within the mobile component.

In another example, a system includes an endoscope having a proximal end with an axial opening, a distal end with a radial opening and a working channel extending therebetween; a guidewire; and a medical device extendable through the working channel and configured to be directed toward an opening of a body lumen. The medical device can be any of those described herein.

Alternatively or additionally to any of the examples above, the guidewire can extend through the elongate tube of the medical device.

Alternatively or additionally to any of the examples above, the steerable distal tip of the medical device can further include a flexible tube connecting a lumen within the base component to a lumen within the mobile component.

Alternatively or additionally to any of the examples above, the guidewire can extend through the lumen within the base component, the flexible tube, and the lumen within the mobile component.

Alternatively or additionally to any of the examples above, the system can further include a power source connected to the wire filament of the medical device.

Alternatively or additionally to any of the examples above, the system can further include a fluid source in communication with the working channel.

Alternatively or additionally to any of the examples above, the system can further include a sensor configured for insertion through the working channel.

Alternatively or additionally to any of the examples above, the handle of the medical device can include a first actuating portion connected to the wire filament.

Alternatively or additionally to any of the examples above, actuation of the first actuating portion of the handle of the medical device in an axial direction can be configured to cause the distal end of the elongate tube to flex and expose a cutting portion of the wire filament.

As another example, a method of accessing a body lumen of a subject can include extending an endoscope having a working channel into the body lumen of the subject; extending an elongate tube having a distal end and a lumen through the working channel; directing the distal end of the elongate tube through an opening of the body lumen; and steering a steerable distal tip forming a terminal end of the elongate tube toward a duct located distal of the opening of the body lumen.

Alternatively or additionally to any of the examples above, steering the steerable distal tip can include axially adjusting a first portion of a handle in communication with a mobile portion of the distal tip to move the mobile portion of the distal tip relative to a base portion of the distal tip to point toward the duct.

Alternatively or additionally to any of the examples above, the method can further include extending a guidewire through the steerable distal tip and into the duct.

Alternatively or additionally to any of the examples above, the method can further include removing the elongate tube from the body lumen and the working channel of the endoscope while maintaining the guidewire in the duct; and inserting another elongate tube over the guidewire and into the duct.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 is a schematic view of an illustrative system for accessing an opening of a body lumen of a subject positioned within the subject;

FIG. 2 is a schematic view of the system depicted in FIG. 1, taken from circle-2;

FIG. 3 is a schematic view of an illustrative system for accessing a body lumen;

FIG. 4 is a schematic cross-sectional view of an illustrative elongate tube of an accessory medical device;

FIGS. 5A-C are schematic views of an illustrative a distal tip of an elongate tube of an accessory medical device;

FIGS. 6A-6D are schematic views of an illustrative base component of the distal tip shown in FIGS. 5A-5C;

FIGS. 7A-7D are schematic views of an illustrative mobile component of the distal tip shown in FIGS. 6A-6D;

FIG. 8 is a schematic view of an illustrative system for accessing a body lumen of a subject with a distal tip of the system positioned proximate an opening of the body lumen; and

FIGS. 9-12 are schematic views of an illustrative system for accessing a body lumen of a subject being used to access the body lumen.

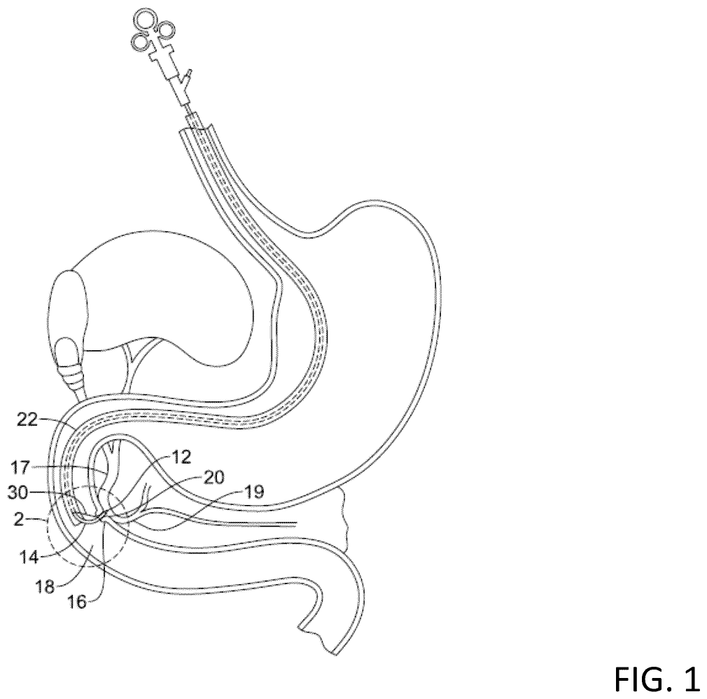

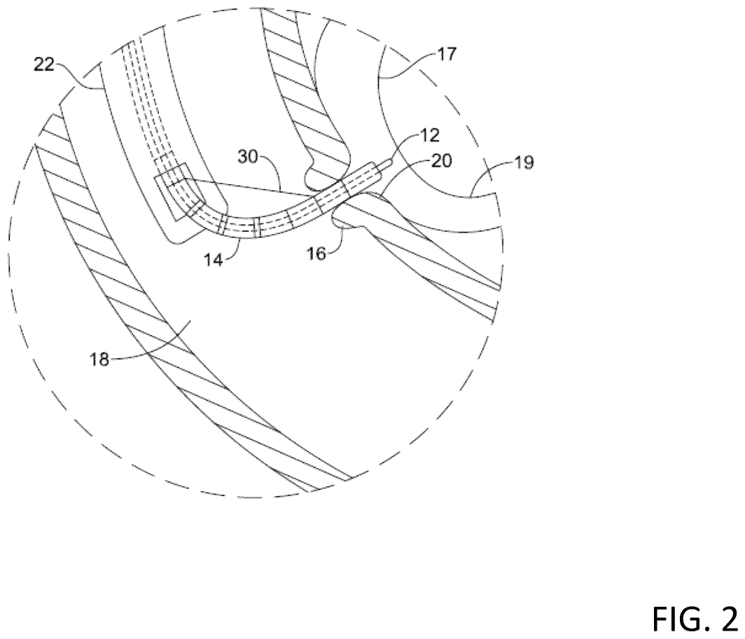

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

As used in this disclosure, the terms “upper” and “lower” are relative terms used to differentiate between opposite directions, not the overall orientation of the device. One of ordinary skill will recognize that a device could operate, for example, with the “uppermost” component at the bottom and the “lowermost” component at the top (i.e., “upside-down” when comparing the device operation to the terms), or in any other absolute orientation relative to the user and/or gravity.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

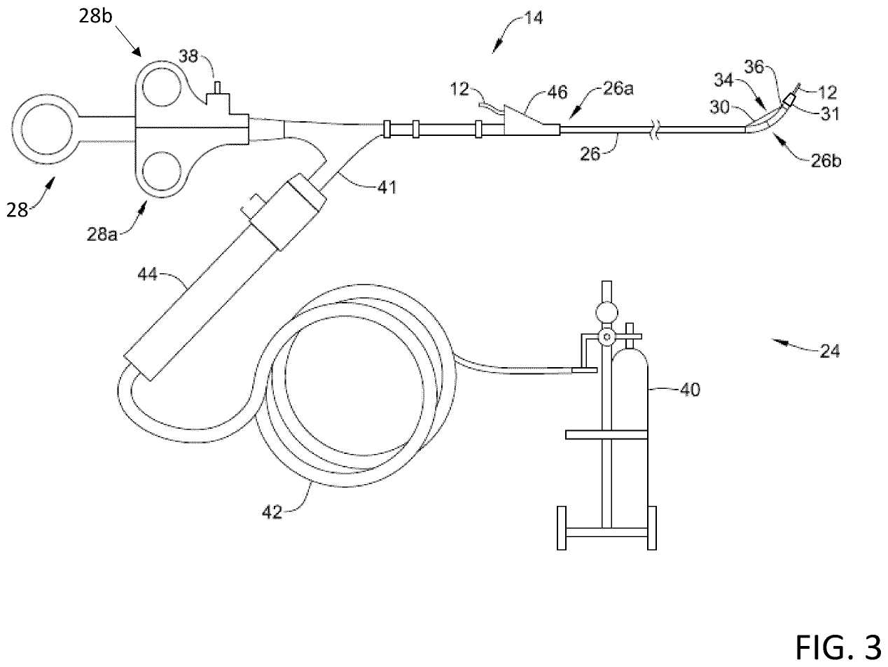

FIG. 3 schematically depicts an illustrative system 24 for accessing a body lumen. In some cases, the system 24 may include, among other components, the accessory medical device 14 with a flexible elongate tube 26. The tube 26 may have a proximal end 26a and a distal end 26b. The distal end 26b of the tube 26 may have a distal tip 31 extending distal of a distal end 36 of the wire filament 30 and may be configured to be directed toward and/or through an opening of a body lumen. The proximal end 26a of the tube 26 may be connected to, coupled with, or otherwise in communication with a handle 28.

The handle 28 may be any suitable type of handle. As schematically depicted in FIG. 3, the handle 28 may have a thumb hole and two finger holes configured to receive fingers from one hand of the user. Each of a first portion 28a and a second portion 28b of a handle may include one of these finger holes to allow for separate operation of the first and second portions 28a and 28b. Other configurations of the first and second portions 28a, 28b of the handle 28 are contemplated. Although not required, the handle 28, as illustrated, may be held and/or operated with a single hand of a user. The first portion 28a can be manipulated to control the wire filament 30, while the second portion 28b can be manipulated to control a steering wire as further described.

The second portion 28b of the handle 28 may be configured to be adjusted or manipulated to control a position of a distal end 26b of the elongate tube 26 and/or the wire filament 30. In some cases, the second portion 28b may be configured to adjust axially or longitudinally and radially or rotationally to adjust a position of the distal end 26b of the elongated tube 26 and/or the wire 30 in a plurality of directions.

A wire filament 30 may extend along the elongate tube 26 and may be energized for the purpose of cutting tissue within a subject. The wire filament 30 may have a proximal end (not shown) connected to an actuating portion 32 (e.g., the second portion 28b) of the handle 28, and a proximal portion extending at least partially within the tube 26. The wire filament 30 also may have a distal portion 34 (e.g., a cutting portion) extending external to the elongate tube 26, and a distal end 36 of the filament 30 may be connected to the distal end 26b of the elongate tube 26.

The wire filament 30 may be received within a lumen of the tube 26 at least along a proximal portion of the filament 30 and may be configured to slide within the lumen. In some cases, the actuating portion 32 of the handle 28 may be coupled to the wire filament 30 and manipulated to slide the wire filament 30 along the tube 26 (e.g., within the lumen of the tube 26). In one example, the actuating portion 32 (e.g., the second portion 28b) of the handle 28 may be longitudinally adjusted relative to the first portion 28a of the handle 28 to apply and remove tension to and/or from the wire filament 30.

Sliding or adjusting the wire filament 30 may result in the distal end 26b of the elongate tube 26 moving responsively to the sliding of the wire filament 30. As such, the actuating portion 32 of the handle 28 may be manipulated to control or adjust a position of the distal end 26b of the elongate tube 26, for example, to direct the distal end 26b toward the opening of a body lumen. In some cases, natural tension in or acting on the tube 26 and/or the wire filament 30 may result in the wire filament 30 returning to a relaxed position or state once a tension applied to the wire filament 30 via the handle 28 is released.

In some cases, the wire filament 30 may be utilized to electrically cut or remove tissue of the subject (e.g., to cut and cauterize tissue) and may be considered a cautery wire. As such, the handle 28 or other suitable portion of the medical device 22 may include an electrical connection 38 for an energy source (e.g., a radiofrequency energy source, or the like) to energize the wire filament 30 and facilitate cutting tissue. When the actuating portion 32 of the handle 28 is manipulated to adjust a position of the distal end 26b of the tube 26 and/or utilize the wire filament 30 for cutting tissue, the wire filament 30 may be spaced from the tube 26 and electrified to facilitate cutting tissue at or adjacent to the opening of the body lumen.

The distal tip 31 of the elongate tube may be flexible and configured to adjust in response to steering with or adjustment of a control configuration at a proximal end of the accessory medical device 14. Although the distal end 26b of the elongate tube 26 of the accessory medical device 14 may be configured to bend or otherwise adjust in response to actuation of the wire filament 30 or in response to being advanced along another device (e.g., an elevator of a duodenoscope and/or other suitable device) that directs a position of the elongated tube 26, such control over or manipulation of the distal end 26b of the elongate tube 26 using the wire filament 30 may be limited to adjustment in the direction of the wire filament 30 or in the direction provided by the distal end 26b being advanced along the other device and lack precise control needed to access a desired body lumen (e.g., a common bile duct 17, a pancreatic duct 19, etc.) To facilitate precisely controlling a distal end of the accessory medical device 14 while the device 14 is within a subject, the distal tip 31 may be configured to be adjustable or steerable in two or more directions through control at the proximal end of the accessory medical device 14 such that distal end 26b of the elongate tube 26 may be guided to a desired body lumen of a subject using finer manipulation of the distal tip 31 than is available via manipulation of the elongate tube using the wire filament 30 and/or interactions with other medical devices. The distal tip 31 may be a steerable distal tip.

The steering wire used to control the distal tip 31 may have a distal end coupled to the distal tip 31, may extend through a lumen of the elongate tube 26 to the handle 28, and may have a proximal end coupled to the actuating portion 32 (e.g., the second portion 28b) of the handle 28. A distal end of the wire may be coupled to the distal tip 31 and/or a proximal end of the wire may be coupled to the actuating portion 32 of the handle 28 in any suitable manner. In one example, the wire may be coupled to the distal tip 31 and/or the actuating portion 32 of the handle 28 using adhesion, for example, through ultraviolet (UV) curing techniques, crimping techniques, and/or other suitable connecting or coupling techniques.

In addition to the steering wire lumen in which the wire for controlling a position of the distal tip 31, when included, extends, the accessory medical device 14 may include one or more other lumens extending from at least the proximal end 26a of the tube 26 to the distal end 26b of the tube 26. In some cases, one or more of the lumens may have a distal opening through the distal tip 31 of the elongate tube 26.

In some cases, a lumen of the one or more lumens of the elongate tube 26 may have a fluid connection at a proximal end for a fluid source (e.g., a pressurized fluid source 40 such as a CO2 tank, a contrast fluid source such as a syringe, etc.) The lumen may be configured to apply a fluid from the fluid source through an opening at the distal end 26b of the elongated tube 26 to a body lumen of the subject.

Although FIG. 3 depicts the fluid source 40 coupled to a fluid port 41 of the accessory medical device 14 via a fluid tube 42 and a flow regulator 44, the fluid source 40 and/or other suitable fluid sources (e.g., syringes, etc.) may be coupled to the fluid port 41 in other suitable manners. In one example, a syringe with contrast fluid may be coupled to the fluid port 41 via a luer lock connection and/or other suitable connection. Contrast agent may be delivered into the body lumen and may be used to temporarily improve imaging of the inside of the body lumen by, for example, x-ray, computed tomography (CT), or magnetic resonance (MR) imaging, ultrasound, and the like. Alternatively, separate lumens and separate fluid ports may be provided for each of a pressurized fluid and a contrast fluid.

A further lumen of the one or more lumens may be configured to accept the guidewire 12 and/or one or more other suitable medical devices usable with the accessory medical device 14. The further lumen may extend from the distal end 26b of the tube 26 (e.g., an opening at the distal tip 31) at least partially along the tube 26 toward the proximal end 26a of the tube 26.

In some cases, the guidewire 12 or other suitable medical devices may extend through the further lumen of the elongate tube 26 and proximally out of an aperture port 46. The aperture port 46 may be located at the distal end of the handle 28 and/or at one or more other suitable locations. In some cases, the aperture port 46 and the tube 26 may be configured so that the guidewire 12 and/or other suitable medical devices may be stripped through the side of the tube 26 for a rapid removal or exchange of devices, but this is not required.

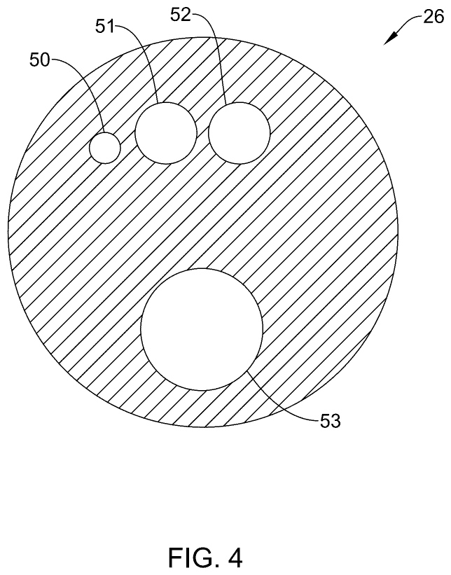

FIG. 4 schematically depicts a cross-sectional view of the elongate tube 26 depicting one example of the one or more lumens extending through the elongate tube 26, taken at a location distal of the aperture port 46 and proximal of the distal end of the distal tip 31. Although other configurations are contemplated, the elongate tube 26 may include a steering wire lumen 50, one or more fluid lumens 51, one or more wire filament lumens 52, and one or more medical device lumens 53. As depicted in FIG. 4, the elongate tube 26 may include a steering wire lumen 50, a fluid lumen 51, where the fluid lumen 51 may be for pressurized fluid and/or for contrast fluid, a wire filament lumen 52 for the wire filament 30 connectable to the electrical connection 38, and a medical device lumen 53 for receiving a guidewire or other suitable medical device.

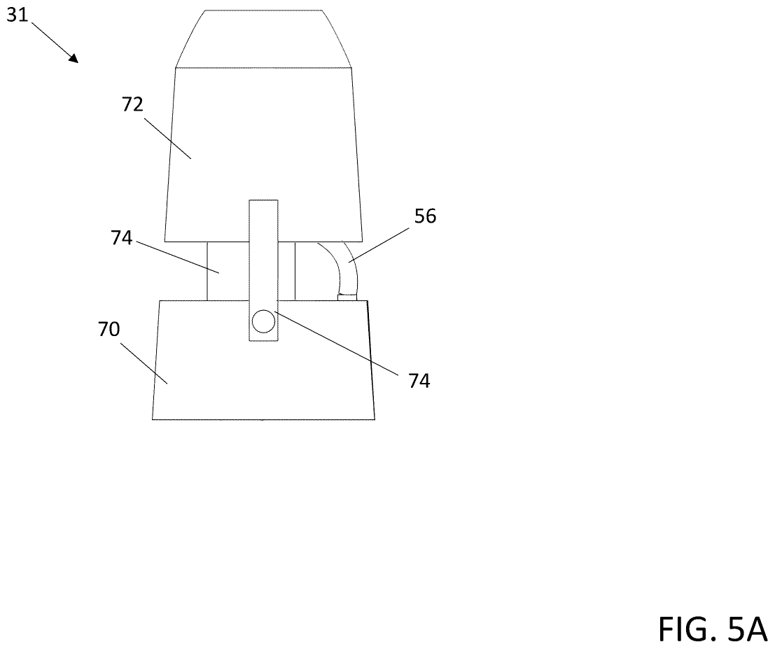

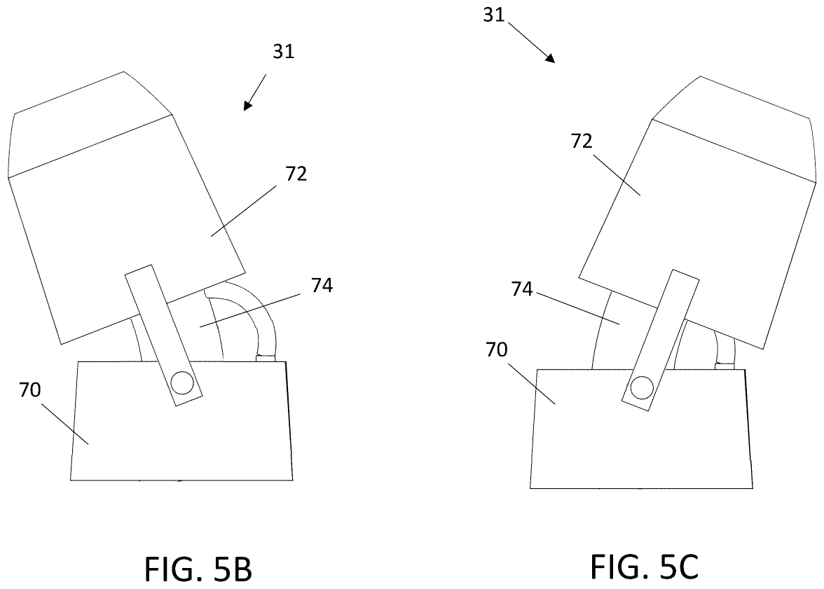

FIGS. 5A-5C depict components of the distal tip 31, including the base component 70 and mobile component 72. A flexible tube 74 stretches between the components 70 and 72. The mobile component 72 includes hinges 76 that anchor on opposite sides of the base component 70. The end of the steering wire 56 is attached to the mobile component 72 for steering the tip 31. FIGS. 5B and 5C illustrate the mobile component 72 angled to the left and to the right relative to the base component 70.

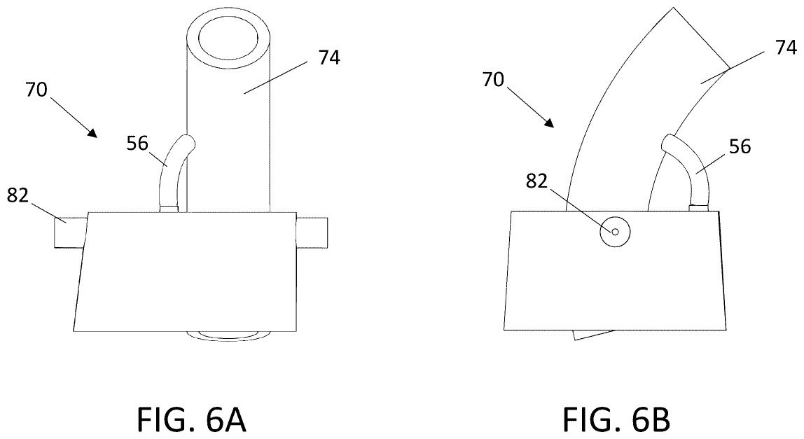

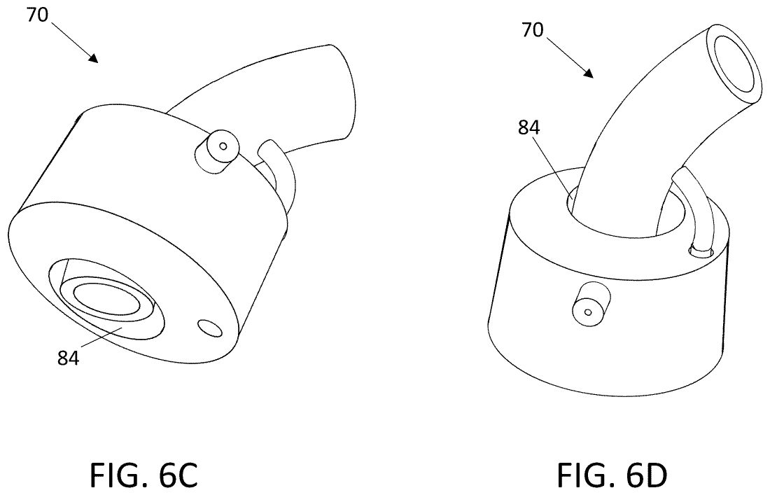

As shown in FIGS. 6A-6D, the base component 70 includes a working lumen 78 to accommodate the flexible tube 74, while a steering wire lumen 80 accommodates the steering wire 56. Hinge knuckles 82 on opposite sides of the base component 70 accommodate the hinges 76. The base component 70 is attached to the distal end of the elongate tube 26 and may be made of a relatively rigid material such as metal or hard plastic. In some implementations, the base component may be formed integrally with the elongate tube 26. In some implementations, the base component 70 may be glued, welded, or otherwise affixed to the elongate tube 26, either removably or permanently. Based on the material properties of the elongate tube 26 and the base component 70, contact between the components at high heat may, in some implementations, be sufficient to adhere the base component 70 to the elongate tube 26.

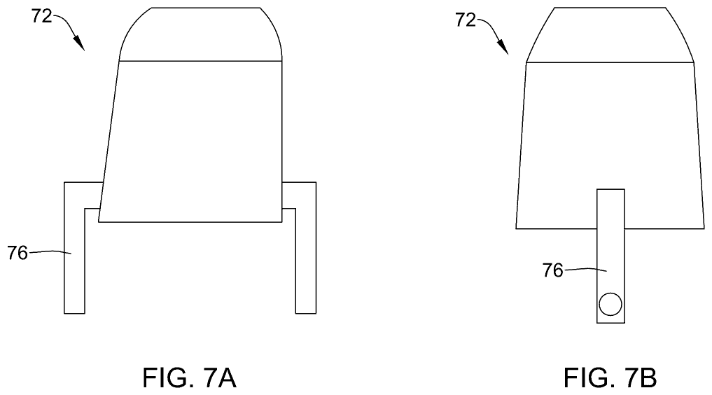

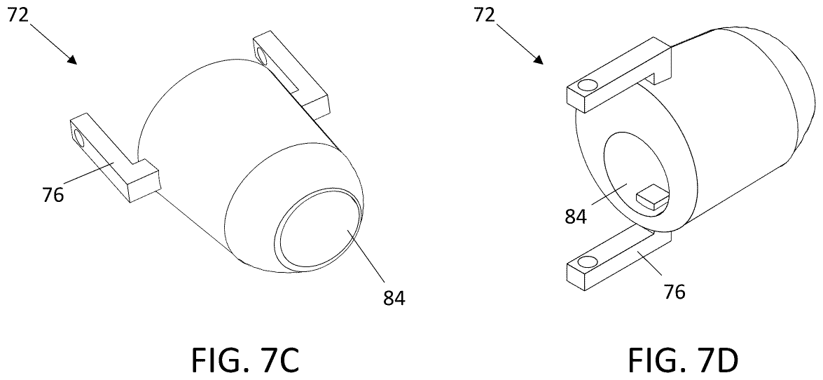

FIGS. 7A-7D depict the mobile component 72, its conical shape aiding insertion. The mobile component 72 is formed of rigid material, such as metal or hard plastic, that may be the same as the rigid material of the base component 70. The hinges 76 may be integral with main body of the mobile component 72 or may be formed separately and attached. A central lumen 84 extends through the main body of the mobile component 72 and accommodates anything passing through the working channel of the endoscope, such as the guidewire.

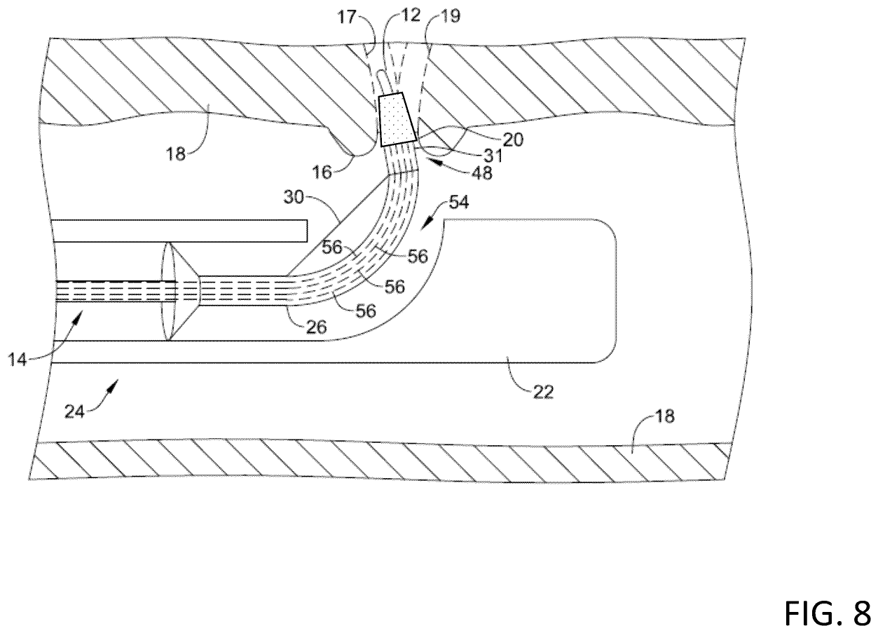

FIG. 8 depicts an enlarged distal end of the system 24 with the medial device 22 positioned adjacent an opening 48 (e.g., an opening defined by the major papilla 16) of a body lumen (e.g., a body lumen defined by the sphincter of Oddi 20). As depicted in FIG. 8, tension has been applied to the wire filament 30 to cause the distal end 26b of the elongate tube 26 to bend in the direction of the wire filament 30 exiting the medical device 22 through an opening 54 thereof. As the distal tip 31 enters through the opening 48 and into a body lumen of the subject, a position of the distal tip 31 of the elongate tube 26 may be adjusted by manipulating (e.g., selectively tensioning or otherwise applying tension to) steering wire 56 and directed to enter the common bile duct 17 as the accessory medical device 14 extends through the medical device 22. However, the steering wire 56 may be manipulated in one or more other suitable manners to direct the distal tip 31 to enter one or more other suitable body lumens (e.g., the pancreatic duct 19, etc.)

In some cases, the steering wire 56 may extend from the distal tip 31 to the handle 28 (e.g., to the actuating portion 32 of the handle 28 and/or other suitable portion of the handles). When so configured, the handle 28 may be adjusted or manipulated to cause or release tension the steering wire 56 to adjust a position of the distal tip 31.

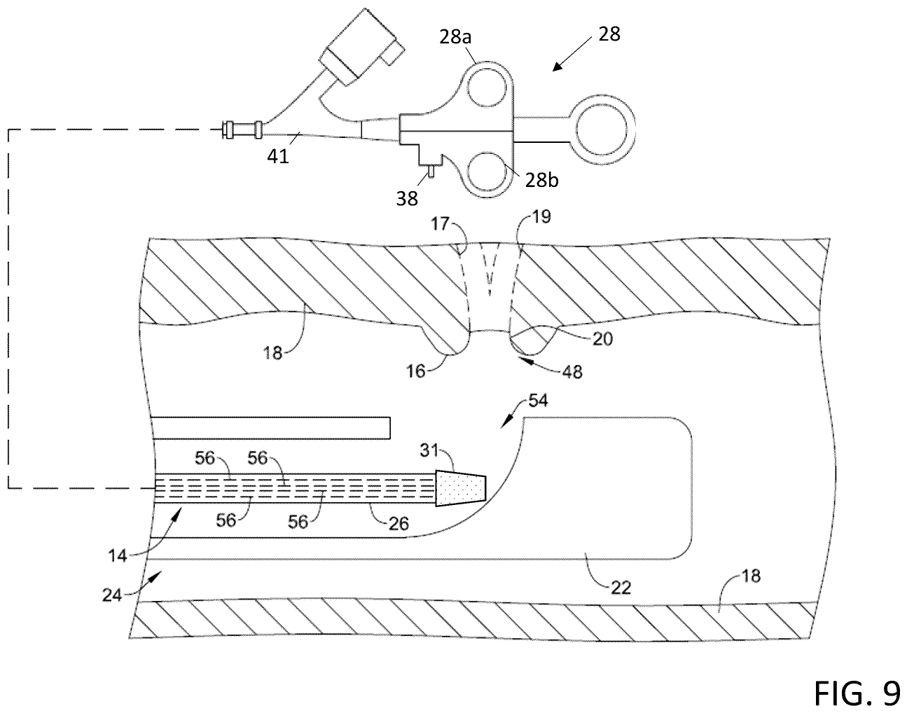

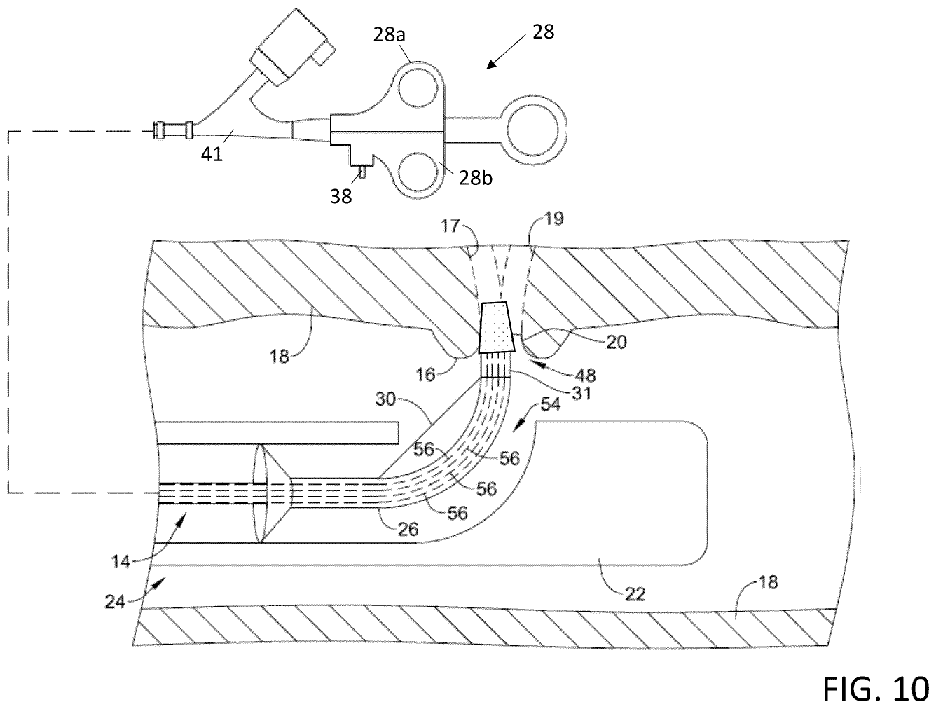

FIGS. 9-12 schematically depict positions of components of the system 24 when controlling the distal end 26b of the accessory medical device 14 by adjustments made at the handle 28 to apply tension to the wire filament 30 and/or the steering wire 56. In FIG. 9, the handle 28 is schematically depicted from a side perspective view. In FIGS. 10-12, the handle 28 is schematically depicted in cross-sectional views to facilitate depicting adjustments or movements of the actuating portion 32 that result in adjusting the distal end 26b of the elongate tube 26.

As depicted in FIG. 9, the system 24 is positioned within a duodenum 18 of a subject and the opening 54 of the medial device 22 has been positioned proximate the papilla of Vater 16 of the subject near (e.g., slightly distal of) the opening 48 of the body lumen. Initially, the distal end 26b of the elongate tube 26 may be positioned within the medical device 22 proximate the papilla 16 with the second portion 28b of the handle 28 (e.g., the actuating portion 32, which is referred to as the second portion 28b of the handle 28 with respect to FIGS. 9-12) in a resting or relaxed position relative to the first portion 28a thereof.

FIG. 10 schematically depicts a tension applied on the wire filament 30 by longitudinally adjusting, in a direction of arrow L, the second portion 28b (e.g., the actuating portion 32) of the handle 28 relative to the first portion 28a, which may cause the distal end 26b of the elongate tube 26 to bend in a direction of the wire filament 30. Prior, while, or after tension is applied to the wire filament 30, the accessory medical device 14 may be advanced in a distal direction relative to the medical device 22, such that a distal end 26b of the elongate tube 26 may exit the opening 54 of the medical device 22 and the distal tip 31 may approach and/or enter the opening 48 at the papilla 16.

As depicted in FIG. 10, a proximal end of the wire filament 30 may be coupled to the stem portion 60 and steering wire 56 may be coupled to a joint 62 of the second portion 28b of the handle 28.

As can be seen in the cross-sectional view of the handle 28 in FIG. 10, a manipulating portion 58 of the second portion 28b of the handle 28 may be coupled to a stem portion 60 of the second portion 28b via the joint 62 (e.g., a rotational joint or other suitable type of joint). When the second portion 28b of the handle 28 is longitudinally adjusted in the direction of arrow L, the joint 62 may facilitate longitudinal adjustment of the manipulating portion 58 and the stem portion 60 together relative to the first portion 28a of the handle 28.

Once the distal tip 31 has entered the opening 48 and/or at one or more other suitable times, the wire filament 30 may be utilized to cut or otherwise remove tissue from around the opening 48. In one example, energy may be applied to the wire filament 30 and the electrified wire filament 30 may be brought into contact with the tissue around the opening 48 by rotating the accessory medical device 14. Other techniques may be utilized to remove tissue around the opening 48 using the wire filament 30.

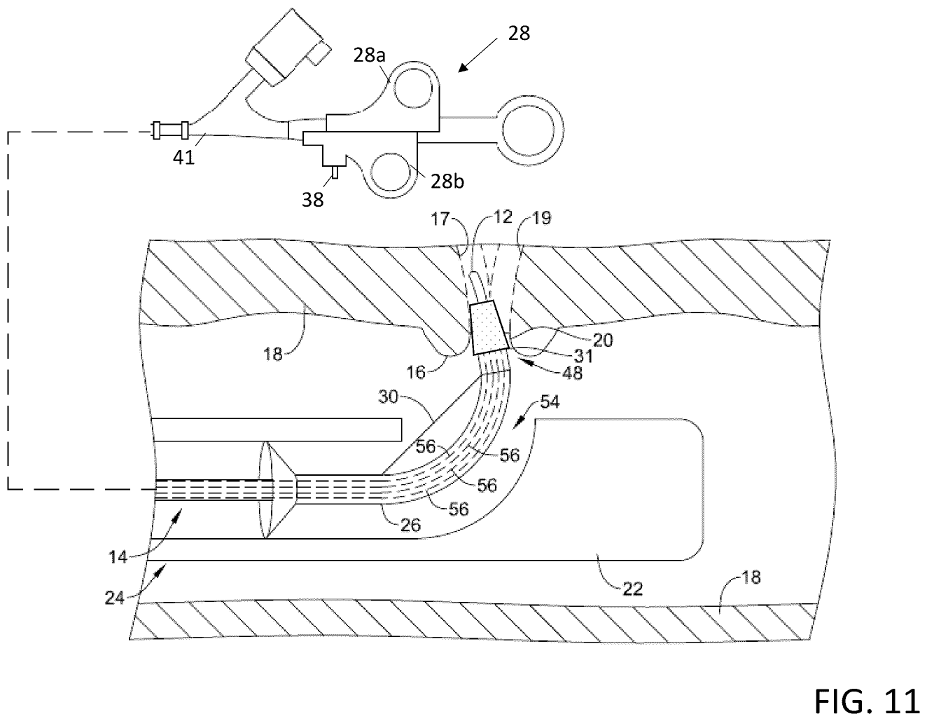

FIG. 11 schematically depicts the accessory medical device 14 with a tension on the wire filament 30 and a manipulating portion 58 of the second portion 28b (e.g., the actuating portion 32) of the handle 28 that is coupled to the steering wire 56, where the manipulating portion 58 may be rotated or manipulated in a radial direction of arrow R with respect to a longitudinal axis of the handle 28 to apply a tension to the steering wire 56. Such rotation and/or radial positioning of the manipulation portion 58 and the associated tension on the steering wire 56 may result in movement of the distal tip 31, as depicted in FIG. 11, to direct the guidewire 12 and the elongate tube 26 into the common bile duct 17. Although the manipulating portion 58 of the second portion 28b of the handle 28 has been depicted as causing the distal tip 31 to adjust toward the common bile duct 17, a user may manipulate the manipulating portion 58 in one or more other suitable manners to direct the distal tip 31 in one or more other directions (e.g., toward the pancreatic duct 19 and/or other suitable ducts). Prior to, while, or after the distal tip 31 is directed toward the common bile duct 17 and/or the manipulating portion 58 is manipulated, the accessory medical device 14 and/or the guidewire 12 may be advanced in a distal direction relative to the medical device 22, such the distal tip 31 and/or the guidewire 12 may approach and/or enter the common bile duct 17.

In some cases, the joint 62 coupling the manipulating portion 58 and the stem 60 may facilitate rotational and/or radial movement of the manipulating portion 58 relative to the stem portion 60, along with the longitudinal movement of the manipulating portion 58 with the stem portion 60 relative to the first portion 28a of the handle 28. Although other joint configurations are contemplated, in one example, the joint 62 between the manipulating portion 58 and the stem portion 60 of the second portion 28b of the handle 28 may be ball-socket joint. As depicted in FIG. 11, the ball 64 of the ball-socket joint may be part of the manipulating portion 58 and in communication with the steering wire 56, while the socket 66 may be part of the stem portion 60, but this is not required and other configurations are contemplated.

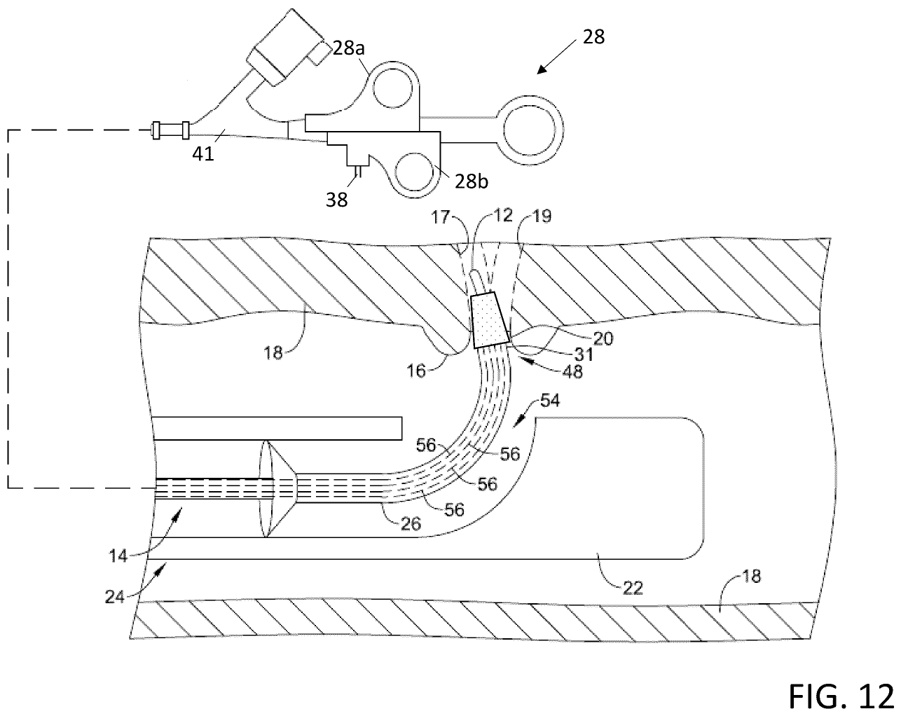

FIG. 12 schematically depicts the accessory medical device 14 with the manipulating portion 58 of the second portion 28b of the handle 28 rotated or manipulated in a radial direction of the arrow R with respect to a longitudinal axis of the handle 28. Further, relative to FIGS. 10 and 11, the longitudinal force on the second portion 28b of the handle 28 in the direction of arrow L has been released and the wire filament 30 has returned to a relaxed state. As a result of the configuration of the second portion 28b of the handle 28, the wire filament 30 may no longer be tensioned and in a cutting position, spaced away from the elongated tube 26, but the distal tip 31 may directed toward the common bile duct 17 due to radial adjustment of the manipulating portion 58 relative to the stem portion 60 of the second portion 28b of the handle 28 in the direction of the arrow R with respect to a longitudinal axis of the handle 28.

In some cases, it may be advantageous to control or adjust the distal tip 31 of the elongate tube 26 individually or separately from the rest of the distal portion 26b of the elongate tube 26. In one example, fine adjustment of the distal tip 31 using the manipulating portion 58 of the handle 28, as discussed herein or otherwise, may allow for directing the accessory medical device 14 and/or the guidewire 12 therein to a body lumen of a subject that is in a direction different than a bend in the elongate tube caused by tensioning the wire filament 30 and/or may facilitate directing or positioning the elongate tube 26 or the guidewire 12 extending therein toward a target body lumen after tension has been removed from the wire filament 30.

In some cases, the elongate tube 26, the wire filament 30, the steering wire 56, and/or other components of the accessory medical device 14 may be configured to return to a relaxed state when forces causing movement of the distal portion 26b of the elongate tube 26 are removed from the handle 28. For example, the elongate tube 26, the wire filament 30, and/or the steering wire 56 may be formed from resilient polymer, metal, and/or combinations of polymer and metal materials that facilitate the wire filament 30 and/or the steering wire 56 returning to the relaxed state.

The materials that can be used for the various components of the systems presently disclosed may include those commonly associated with medical devices. For simplicity purposes, the following discussion makes reference to system 24 referenced above. However, this is not intended to limit the devices and methods described herein, as the discussion may be applied to other similar devices and/or components of devices disclosed herein.

The system 24 and/or components thereof may be made from a metal, metal

alloy, polymer (some examples of which are disclosed below), a metal-polymer composite, ceramics, combinations thereof, and the like, or other suitable material. Some examples of suitable metals and metal alloys include stainless steel, such as 304V, 304 L, and 316 L V stainless steel; mild steel; nickel-titanium alloy such as linear-elastic and/or super-elastic nitinol; other nickel alloys such as nickel-chromium-molybdenum alloys (e.g., UNS:N06625 such as INCONEL® 625, UNS:N 06022 such as HASTELLOY® C-22®, UNS:N10276 such as HASTELLOY® C276®, other HASTELLOY® alloys, and the like), nickel-copper alloys (e.g., UNS: N 04400 such as MONEL® 400,NICKELVAC® 400, NICORROS® 400, and the like), nickel-cobalt-chromium-molybdenum alloys (e.g., UNS:R30035 such as M P35-N® and the like), nickel-molybdenum alloys (e.g., UNS:N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS:R30003 such as ELGILOY®, PHY NOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTA MID® available from Elf A tochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PV dC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, a degree of Magnetic Resonance Imaging (MRI) compatibility is imparted into the system 24. For example, the system 24 or portions thereof may be made of a material that does not substantially distort the image and create substantial artifacts (i.e., gaps in the image). Certain ferromagnetic materials, for example, may not be suitable because they may create artifacts in an MRI image. The system 24 or portions thereof may also be made from a material that the MRI machine can image. Some materials that exhibit these characteristics include, for example, tungsten, cobalt-chromium-molybdenum alloys (e.g., UNS:R0003 such as ELGILOY®, PHY NOX®, and the like), nickel-cobalt-chromium-molybdenum alloys (e.g., UNS:R30035 such as MP35-N® and the like), nitinol, and the like, and others.

As alluded to above, the tubular and/or elongated components of the system 24 may include one or more tubular members that may have slots formed therein. Various embodiments of arrangements and configurations of slots are contemplated. For example, in some embodiments, at least some, if not all of the slots are disposed at the same or a similar angle with respect to the longitudinal axis of the tubular and/or elongated components of the system 24. The slots can be disposed at an angle that is perpendicular, or substantially perpendicular, and/or can be characterized as being disposed in a plane that is normal to the longitudinal axis of the tubular and/or elongated components of the system 24. However, in other embodiments, the slots can be disposed at an angle that is not perpendicular, and/or can be characterized as being disposed in a plane that is not normal to the longitudinal axis of the tubular components of the system 24. Additionally, a group of one or more the slots may be disposed at different angles relative to another group of one or more the slots. The distribution and/or configuration of the slots can also include, to the extent applicable, any of those disclosed in U.S. Pat. No. U.S. Pat. No. 7,914,467, the entire disclosure of which is herein incorporated by reference. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing the system 24 may include forming the slots in the tubular or elongated components of the system 24 using these or other manufacturing steps.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device comprising:
an elongate tube having a proximal end and a distal end configured to be directed toward an opening of a body lumen;
a wire filament extending along the elongate tube, a distal end of the wire filament connected to the distal end of the elongate tube, a proximal portion of the wire filament extending at least partially along the elongate tube, and a distal portion of the wire filament extending external to the elongate tube;
a steerable distal tip extending distal of a distal end of the wire filament, the distal tip comprising a base component and a mobile component distal from the base component;
a handle in mechanical communication with the steerable distal tip; and
a steering wire extending from the handle to the mobile component of the steerable distal tip, such that adjustment of the handle causes tension in the steering wire to adjust the mobile portion of the distal tip relative to the base component of the distal tip.

2. The medical device of claim 1, wherein the handle includes a first actuating portion connected to the wire filament.

3. The medical device of claim 2, wherein actuation of the first actuating portion in an axial direction is configured to cause the distal end of the elongate tube to flex and expose a cutting portion of the wire filament.

4. The medical device of claim 1, wherein the handle includes a second actuating portion connected to the steering wire.

5. The medical device of claim 4, wherein actuation of the actuating portion in an axial direction adjusts the position of the mobile portion of the distal tip relative to the base component of the distal tip.

6. The medical device of claim 1, wherein:
the steering wire extends through at least a portion of the elongate tube; and
the elongate tube includes a first lumen for receiving the wire filament and a second lumen configured to receive the steering wire.

7. The medical device of claim 1, wherein adjustment of the handle in a radial direction relative to the elongate tube is configured to adjust the position of the steerable distal tip.

8. The medical device of claim 1, wherein the steerable distal tip further comprises a flexible tube connecting a lumen within the base component to a lumen within the mobile component.

9. A system, comprising:
an endoscope having a proximal end with an axial opening, a distal end with a radial opening and a working channel extending therebetween;
a guidewire; and
a medical device extendable through the working channel and configured to be directed toward an opening of a body lumen, the medical device comprising:
an elongate tube having a proximal end and a distal end configured to extend through the radial opening;
a steerable distal tip configured to extend through the radial opening, the distal tip comprising a base component and a mobile component distal from the base component;
a handle in mechanical communication with the steerable distal tip; and
a steering wire extending from the handle to the mobile component of the steerable distal tip, such that adjustment of the handle causes tension in the steering wire to adjust the mobile portion of the distal tip relative to the base component of the distal tip.

10. The system of claim 9, wherein the guidewire extends through the elongate tube of the medical device.

11. The system of claim 9, wherein the steerable distal tip of the medical device further comprises a flexible tube connecting a lumen within the base component to a lumen within the mobile component.

12. The system of claim 10, wherein the guidewire extends through the lumen within the base component, the flexible tube, and the lumen within the mobile component.

13. The system of claim 9, further comprising a power source connected to the wire filament of the medical device.

14. The system of claim 9, further comprising a fluid source in communication with the working channel.

15. The system of claim 9, further comprising a sensor configured for insertion through the working channel.

16. The system of claim 9, wherein the handle of the medical device includes a first actuating portion connected to the wire filament, and
wherein actuation of the first actuating portion of the handle of the medical device in an axial direction is configured to cause the distal end of the elongate tube to flex and expose a cutting portion of the wire filament.

17. A method of accessing a body lumen of a subject, comprising:
extending an endoscope having a working channel into the body lumen of the subject;
extending an elongate tube having a distal end and a lumen through the working channel;
directing the distal end of the elongate tube through an opening of the body lumen; and
steering a steerable distal tip forming a terminal end of the elongate tube toward a duct located distal of the opening of the body lumen.

18. The method of claim 17, wherein steering the steerable distal tip comprises axially adjusting a first portion of a handle in communication with a mobile portion of the distal tip to move the mobile portion of the distal tip relative to a base portion of the distal tip to point toward the duct.

19. The method of claim 17, further comprising:
extending a guidewire through the steerable distal tip and into the duct.

20. The method of claim 19, further comprising:
removing the elongate tube from the body lumen and the working channel of the endoscope while maintaining the guidewire in the duct; and
inserting another elongate tube over the guidewire and into the duct.
